(19) United States
(12) Patent Application Publication
(10) Pub. No.: US 2025/0009911 A1
(43) Pub. Date: Jan. 9, 2025

(54) DOTA-HYNIC-iPSMA AS A THERAPEUTIC RADIOPHARMACEUTICAL TARGETING PROSTATE-SPECIFIC MEMBRANE ANTIGEN

(71) Applicants: INSTITUTO NACIONAL DE INVESTIGACIONES NUCLEARES, Ocoyoacac (MX); Telix Innovations SA, Herstal (BE)

(72) Inventors: Guillermina FERRO FLORES, Ocoyoacac (MX); Blanca Elí OCAMPO GARCÍA, Ocoyoacac (MX); Myrna Alejandra LUNA GUTIÉRREZ, Ocoyoacac (MX); Clara Leticia SANTOS CUEVAS, Ocoyoacac (MX); Erika Patricia AZORÍN VEGA, Ocoyoacac (MX); Nallely Patricia JIMÉNEZ MANCILLA, Ocoyoacac (MX); Tania HERNÁNDEZ JIMÉNEZ, Ocoyoacac (MX); Flor de María RAMÍREZ DE LA CRUZ, Ocoyoacac (MX); Khaled Ahmed Adly Ibrahim ATTIA, Herstal (BE)

(73) Assignees: INSTITUTO NACIONAL DE INVESTIGACIONES NUCLEARES, Ocoyoacac (MX); Telix Innovations SA, Herstal (BE)

(21) Appl. No.: 18/886,598

(22) Filed: Sep. 16, 2024

Related U.S. Application Data
(63) Continuation-in-part of application No. 16/980,252, filed on Sep. 11, 2020, now Pat. No. 12,128,114.

(30) Foreign Application Priority Data
Mar. 14, 2018 (MX) MX/A/2018/003175

(51) Int. Cl. A61K 51/04; A61P 35/00

(52) U.S. Cl. CPC A61K 51/0455; A61P 35/00; A61K 2121/00; A61K 2123/00

(57) ABSTRACT

The invention relates to a new therapeutic radiopharmaceutical as an inhibitor of prostate-specific membrane antigen (iPSMA), wherein 1,4,7,10-tetraazacyclododecane-N, N′, N″, N″″ -tetraacetic acid (DOTA) bonded to the heterocyclic molecule hydrazinonicotinamide (HYNIC), generates a rigid chemical structure that minimises the number of conformers and intramolecular hydrogen bonds, thereby producing a favourable spatial orientation of the active site (Lys(Nal)—NH—CO—NH-Glu) in the molecule, for biological recognition by the PSMA protein. The new DOTA-HYNIC-iPSMA radiopharmaceutical accumulates, with high affinity in vivo, in tumours that overexpress the PSMA protein, acting as a radiotherapeutic or radiodiagnostic agent. The purpose of the invention is to provide a new specific radiopharmaceutical (molecular target radiopharmaceutical) for the detection and treatment of tumours with PSMA overexpression. Radiolabelling of the new molecule is demonstrated with lutetium-177, actinium-225, terbium-161, gallium-67 or gallium-68 isotopes.

### CROSS-REFERENCE TO RELATED APPLICATIONS

This application is a continuation-in-part of U.S. patent application Ser. No. 16/980,252, filed Sep. 11, 2020, now allowed, which is a U.S. national stage entry under 5 U.S.C. § 371 of PCT International Patent Application No. PCT/MX2019/000025, filed Mar. 7, 2019, which claims priority to Mexican Patent Application No. MX/a/2018/003175, filed Mar. 14, 2018, the contents of each of which are incorporated herein by reference in their entirety.

### TECHNICAL FIELD OF THE INVENTION

The present invention relates to a novel therapeutic or diagnostic radiopharmaceutical as a prostate-specific membrane antigen inhibitor (iPSMA) wherein 1,4,7,10-tetraazacyclododecano-N, N′, N″, N″″ -tetraacetic acid (DOTA) bound to the molecule hydrazinonicotinamide (HYNIC), which is heterocyclic in nature, generates a rigid chemical structure that minimises the number of conformers and intramolecular hydrogen bonds, thereby resulting in a spatial orientation of the active site (Lys (Nal)—NH—CO—NH-Glu) in the molecule that favors biological recognition of the PSMA protein. The novel radiopharmaceutical or radiodiagnostic DOTA-HYNIC-iPSMA accumulates in tumors overexpressing the protein PSMA with high affinity in vivo, thereby acting as radiotherapeutic or radiodiagnostic agent. The molecule can be radiolabelled with lutetium-177, actinium-225, or with terbium-161 for therapeutic purposes, or with gallium-67 or -68 for diagnostic and imaging purposes.

### BACKGROUND

Prostate cancer (PC) is the second most common cancer amongst males worldwide [Jemal A, et al. Cancer statistics, 2010. CA Cancer J Clin. 2010, 60:277-300]. In patients with localised PC, the five-year survival rate is close to 100%, whereas in patients with metastasis, the five-year survival rate is 31% [Wei Q, et al. Global analysis of differentially expressed genes in androgen-independent prostate cancer. Prostate Cancer Prostatic Dis. 2007, 10:167-174]. Almost all patients with metastasis respond well to anti-androgen treatments initially. However, the main cause of death in PC patients is progression to androgen independence.

The enzyme glutamate carboxypeptidase II, also known as prostate-specific membrane antigen (PSMA) is expressed in epithelial cells in the prostate and is highly overexpressed in 95% of advanced prostate cancers. PSMA expression levels are directly correlated with the androgen independence, metastasis and progression of PC [Santoni M., et al. Targeting prostate-specific membrane antigen for personalized therapies in prostate cancer: morphologic and molecular backgrounds and future promises. J Biol Regul Homeost Agents. 2014, 28:555-563] As such, PSMA is an appropriate molecular target for the image-based detection and radiotherapy of metastatic prostate cancer using specific radiopharmaceuticals.

The PSMA gene comprises 19 exons representing approximately 60 kb of genomic DNA. This gene codes for a type II transmembrane protein with a short cytoplasmic fragment (19 amino acids), a hydrophobic transmembrane domain (24 amino acids), and a large extracellular domain (707 amino acids). PSMA contains Zn at the active center of the enzyme, therefore the sequence Glu—NH—CO—NH-Lys (β-naphthyl alanine)=Glu—NH—CO—NH-Lys (Nal) has been proposed as an effective inhibitor of the activity thereof [Benesová, M, et al., Preclinical evaluation of a tailor-made DOTA-conjugated PSMA inhibitor with optimized linker moiety for imaging and endoradiotherapy of prostate cancer. J Nucl Med, 56, 2015:914-920]. In the specific chemical interaction, the three carboxyl groups of the Glu—NH—CO—NH-Lys fragment interact electrostatically with the peptide side-chains at the active site of PSMA, the urea oxygen coordinates to zinc, and the aromatic structure in Nal interacts to ensure coupling with the active hydrophobic site in the enzyme. Recent clinical studies have shown that the application of two different PSMA inhibitor derivatives labeled with Lu-177, namely ¹⁷⁷Lu-PSMA-617 and ¹⁷⁷Lu-PSMA-I&T, results in a significant reduction in prostate antigen (PSA) levels in 50-70% of PC patients, with no severe side-significantly increasing patient effects, thereby survival [Ahmadzadehfar H., et al. Early side effects and first results of radioligand therapy with ¹⁷⁷Lu-DKFZ-617 PSMA of castrate-resistant metastatic prostate cancer: a two-centre study. EJNMMI Res. 2015. 5:36; Kratochwil C et al. [¹⁷⁷Lu]Lutetium-labelled PSMA ligand-induced remission in a patient with metastatic prostate cancer. Eur J Nucl Med Mol Imaging, 42, 2015:987-988; Weineisen M. et al., ⁶⁸Ga- and ¹⁷⁷Lu-Labeled PSMA I&T: Optimization of a PSMA-Targeted Theranostic Concept and First Proof-of-Concept Human Studies. J Nucl Med 2015; 56:1169-1176; Baum, R. P., et al. Lutetium-177 PSMA radioligand therapy of metastatic castration-resistant prostate cancer: safety and efficacy. J Nucl Med, 2016, 57:1006-1013; Kratochwil, C, et al. PSMA-targeted radionuclide therapy of metastatic castration-resistant prostate cancer with Lu-177 labeled PSMA-617. J Nucl Med, 2016, 57:1170-1176; Rahbar, K et al. Response to and tolerability of a single dose of ¹⁷⁷Lu-PSMA-617 in patients with metastatic castration-resistant prostate cancer: a multicenter retrospective analysis. J Nucl Med, 2016, 57:1334-1338; Rahbar, K et al. German Multicenter Study Investigating ¹⁷⁷Lu-PSMA-617 Radioligand Therapy in Advanced Prostate Cancer Patients. J Nucl Med, 2017, 58:85-90]. The PSMA protein is multifunctional as it can act as an internalization receptor, a nutrient absorption enzyme, or as a peptidase that plays a role in signal transduction in epithelial cells and in cell migration [Rajasekaran A. et al. Is prostate-specific membrane antigen a multifunctional protein? American Journal of Physiology-Cell Physiology. 2005, 288: C975-C981]. As such, PSMA-inhibiting radiopharmaceuticals may also be used in neoplasms other than PC, for example in metastatic breast cancer, osteosarcoma, glioma and differentiated thyroid cancer, amongst others [la Fougére, et al. In vivo visualization of prostate-specific membrane antigen in glioblastoma.Eur J Nucl Med and Mol Imaging, 2015, 42: 170-171; Verburg F A, et al. First evidence of PSMA expression in differentiated thyroid cancer using [⁶⁸Ga] PSMA-HBED-CC PET/CT Eur J Nucl Med and Mol imaging, 2015, 42:1622-1623; Zeng C et al. Prostate-specific membrane antigen: new a potential marker prognostic of osteosarcoma. Medical Oncology, 2012, 29:2234-2239; Sathekge M el al, ⁶⁸Ga-PSMA imaging of metastatic breast cancer. Eur J Nucl Med and Mol Imaging, 2015, 42:1482-1483.

However, prior to any radiotherapy treatment, uptake of the radiopharmaceutical in tumors or their metastases must be evaluated by nuclear imaging in order to confirm whether treatment will be useful for the patient or not and to determine the activity that will need to be administered to provide the ablative dose of radiation to the tumors, in other words personalised and theranostic medicine is applied. To that end, diagnostic PSMA-inhibiting radiopharmaceuticals must be used to obtain molecular images by positron-emission tomography (PET) or single-photon emission computed tomography (SPECT), with ⁶⁸Ga-PSMA-11 (PET) being the most widely used in clinical practice in this regard due to its high sensitivity and specificity [Eder M et al. Novel preclinical and radiopharmaceutical aspects of [68Ga]Ga-PSMA-HBED-CC: a new PET tracer for imaging of prostate cancer. Pharmaceuticals, 2014, 7:779-796; Eder M et al. 68Ga-complex lipophilicity and the targeting property of a urea-based PSMA inhibitor for PET imaging. Bioconjugate Chem, 2012, 23:688-697; Weineisen et al. ⁶⁸Ga-and 177Lu-labeled PSMA I&T: optimization of a PSMA-targeted theranostic concept and first proof-of-concept human studies.” J Nucl Med, 2015, 56:1169-1176; Afshar-Oromieh, A., et al. Comparison of PET/CT and PET/MRI hybrid systems using ⁶⁸Ga-labeled PSMA ligand for the diagnosis of recurrent prostate cancer: initial experience. Eur J Nucl Med and Molecular Imaging 41.5 (2014); 887-897].

However, both nationally and internationally, SPECT studies represent more than 70% of all studies carried out in nuclear medicine given their lower cost and the greater availability of equipment and radionuclides as there is no need for a cyclotron in hospitals or close to them. The most widely used radionuclide for SPECT imaging is ^{99m}Tc and, recently, ^{99m}Tc EDDA/HYNIC-iPSMA was reported as a radiopharmaceutical inhibitor of prostate-specific membrane antigen (iPSMA) containing hydrazinonicotinamide (HYNIC) as a critical chemical group for enhancing the lipophilicity of the molecule for coupling to the hydrophobic sites in PSMA, combined with the conventional use of HYNIC as a chelating agent for the radiometal ^{99m}Tc, with ethylenediaminoacetic acid (EDDA) being used to complete the coordination sphere of the radiometal. The radiopharmaceutical ^{99m}Tc-EDDA/HYNIC/iPSMA detects the PSMA protein overexpressed in prostate cancer cells with high in vivo affinity using SPECT molecular imaging techniques in nuclear medicine [Ferro-Flores G., et al. Clinical translation of a PSMA inhibitor for ^{99m}Tc-based SPECT. Nucl Med Bici, 2017, 48:36-44; Santos-Cuevas et al, ^{99m}Tc-EDDA/HYNIC-iPSMA: Biokinetics and Radiation Dosimetry in Healthy Subjects and Tumor Imaging in Patients with Prostate Cancer. Nucl Med Biol, 2017, 52:1-6; Lawal I. O., et al. Diagnostic sensitivity of Tc-99m HYNIC PSMA SPECT/CT in prostate carcinoma: A comparative analysis with Ga-68 PSMA PET/CT, The Prostate, 2017, 1-8; Ferro-Flores G., et al. ^{99m}Tc-EDDA/HYNIC-iPSMA as a radiopharmaceuticai for detecting the overexpression of prostate-specific membrane antigen, WO2017222362, PCT/MX2017/000068].

In order to develop a theranostic pair for the ligand HYNIC-iPSMA that can be labeled with Lu-177, herein we propose to bind the molecule DOTA to HYNIC, thus generating a rigid chemical structure that minimises the number of conformers and intramolecular hydrogen bonds, thereby resulting in a spatial orientation of the active site (Lys (Nal)—NH—CO—NH-Glu) in the molecule that favors biological recognition of the PSMA protein.

### SUMMARY OF THE INVENTION

Aspect 1. A radiopharmaceutical with the chemical formula DOTA-HYNIC-iPSMA defined by structure (I):

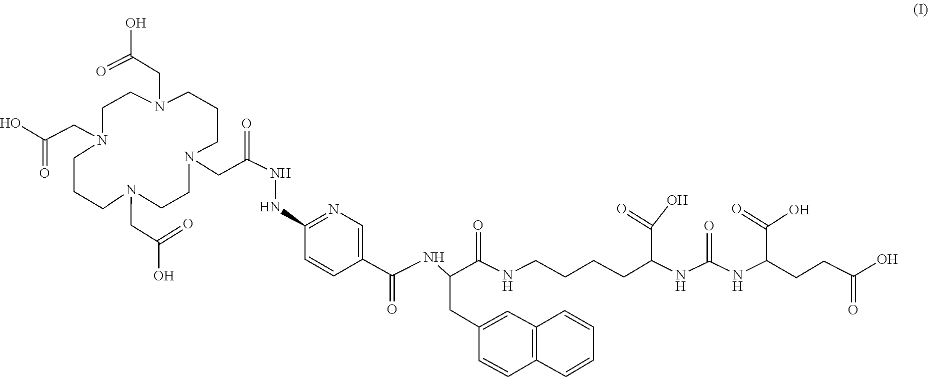

In any of the embodiments herein, said DOTA-HYNIC-panPSMA can be formulated in combination with mannitol and/or ascorbic acid. For example, the DOTA-HYNIC-panPSMA (0.6 mg) can be formulated as a lyophilised pharmaceutical form containing from 40 to 60 mg mannitol and from 10 to 100 mg ascorbic acid, more preferably from 40 to 100 mg or from 40 to 60 mg ascorbic acid.

Aspect 2. The radiopharmaceutical according to aspect 1, radiolabelled with Lutetium-177 defined by structure (II):

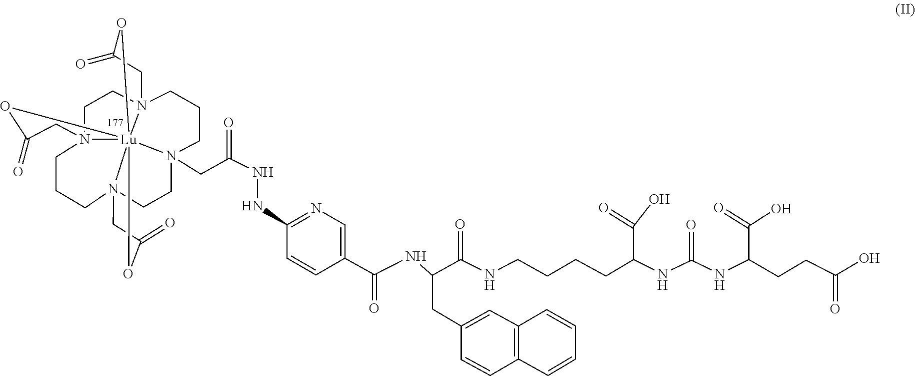

Aspect 3. The radiopharmaceutical according to aspect 1, radiolabelled with actinium-225 defined by structure (III):

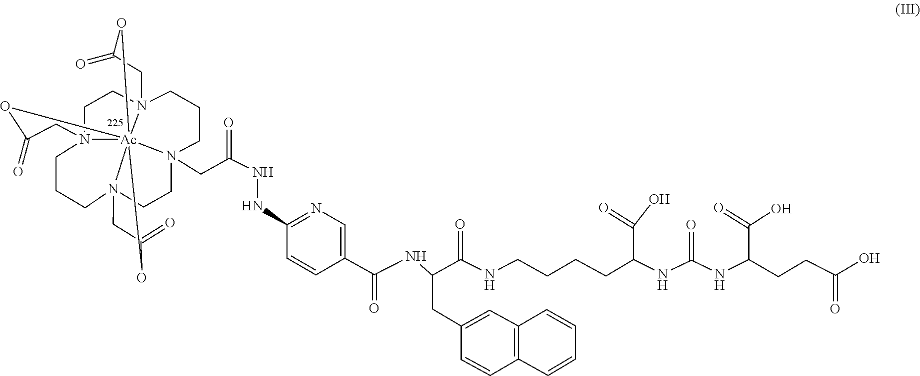

Aspect 4. The radiopharmaceutical according to aspect 1, radiolabelled with terbium-161 defined by structure (IV):

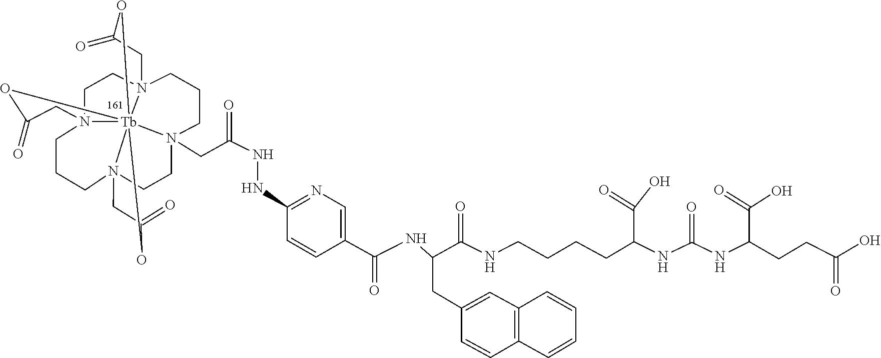

Aspect 5. The radiopharmaceutical according to aspect 1, radiolabelled with gallium-68 defined by structure (V):

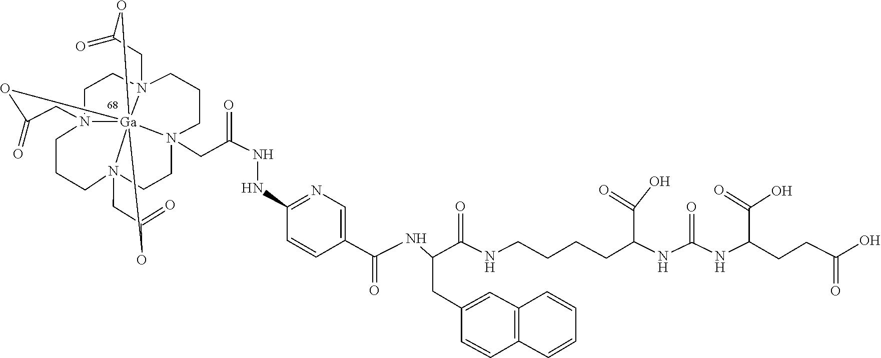

Aspect 6. A radiopharmaceutical composition characterised by comprising a radiopharmaceutical such as that claimed in aspect 1.

Aspect 7. A radiopharmaceutical such as that claimed in aspect 1 for use as a radiotherapeutic agent in tumors that overexpress the protein PSMA (prostate-specific membrane antigen).

Aspect 8. The radiopharmaceutical according to aspect 7, wherein said cancer is selected from the group consisting of: prostate cancer, salivary gland cancer, glioblastoma, thyroid cancer, clear cell renal carcinoma, renal cell carcinoma, hepatocellular carcinoma, lung cancer, adenoid cystic carcinoma of the parotid, and breast cancer.

Aspect 9. An imaging method, comprising the steps of:
i) providing the DOTA-HYNIC-iPSMA radiopharmaceutical according to aspect 1 radiolabelled with lutetium-177, actinium-225, terbium-161, or gallium-68,
ii) administering to a subject a diagnostic amount of said radiolabeled targeting agent; and,
iii) detecting said radiolabeled radiopharmaceutical when bound to its target PSMA using PET, SPECT, PET/CT or SPECT/CT imaging methods.

Aspect 10. The imaging method according to aspect 9, wherein said radiolabel is lutetium-177 and wherein the imaging method used is SPECT or SPECT/CT imaging.

Aspect 11. The imaging method according to aspect 9, wherein said radiolabel is actinium-225 and wherein the imaging method used is SPECT or SPECT/CT imaging.

Aspect 12. The imaging method according to aspect 9, wherein said radiolabel is terbium-161 and wherein the imaging method used is PET, PET/CT, SPECT or SPECT/CT imaging, preferably SPECT or SPECT/CT imaging.

Aspect 13. The imaging method according to aspect 9, wherein said radiolabel is gallium-68 and wherein the imaging method used is PET or PET/CT imaging.

Aspect 14. A method of detecting a tumour or cancer, comprising the steps of:
i) providing a DOTA-HYNIC-iPSMA radiopharmaceutical according to aspect 1 radiolabeled with lutetium-177, actinium-225, terbium-161, or gallium-68,
ii) administering to a subject a diagnostic amount of said radiolabeled DOTA-HYNIC-iPSMA; and,
iii) detecting binding of said radiolabeled DOTA-HYNIC-iPSMA to its PSMA target using PET, SPECT, PET/CT, or SPECT/CT imaging methods.

Aspect 15. The method according to aspect 14, wherein said detection is used for:
(i) initial staging of prostate cancer into intermediate, unfavourable, high, or very high-risk prostate cancer;
(ii) detecting suspected recurrence of prostate cancer and/or detection of metastasis;
(iii) selection for radiotherapeutic treatment such as with Lutetium (177Lu) vipivotide tetraxetan (Pluvicto);
(iv) monitoring prostate cancer for progression into Non-Metastatic or Metastatic Castration-Resistant Prostate Cancer (nmCRPC or mCRPC);
(v) determining response to (radio) therapy;
(vi) real-time imaging or diagnosis in a subject, such as for use in PET scans for tracking slow biological processes or progression of those over a period of time, optionally wherein the real-time imaging or diagnosis is carried out by a computer or processor and hence is directed by a system for in vivo diagnosis or for in vivo imaging in a subject, said system comprising a processing means (processor) configured to carry out the steps of receiving real-time imaging data and presenting said imaging data in real-time to a user, thereby enabling in vivo imaging or in vivo diagnosis of said subject; or
(vii) personalised medicine.

Aspect 16. The method according to aspect 14, wherein said detection method is used to replace the need for taking a prostate biopsy or is used in SPECT, SPECT/CT, PET or PET/CT scan with MRI in clinically significant or intermediate favourable prostate cancers, or in MRI for active surveillance of prostate cancer.

Aspect 17. A method for treatment of cancer, comprising the step of administering an effective amount of DOTA-HYNIC-iPSMA according to aspect 1, radiolabelled with lutetium-177, actinium-225 or terbium-161 to a patient in need thereof, preferably wherein said cancer has been detected using the method of aspect 14.

Aspect 18. The method according to aspect 17, wherein said cancer is over-expressing PSMA.

Aspect 19. The method according to aspect 17 or 18, wherein said cancer is selected from the group consisting of prostate cancer, salivary gland cancer, glioblastoma, thyroid cancer, clear cell renal carcinoma, renal cell carcinoma, hepatocellular carcinoma, lung cancer, adenoid cystic carcinoma of the parotid, and breast cancer.

### BRIEF DESCRIPTION OF THE DRAWINGS

FIG. 1. Chemical structure of Lu-177 radiolabelled DOTA-Hynic-iPSMA.

FIG. 2. Mass spectrum analysis of the Glu—NH—CO—NH-Lys (β-naphthyl alanine)-HYNIC-DOTA (DOTA-HYNIC-iPSMA).

FIG. 3. Radio-chromatogram of ¹⁷⁷Lu-DOTA-HYNIC-iPSMA showing a radiochemical purity of more than 98%, as determined by reverse-phase HPLC.

FIG. 4. SPECT image for the radiopharmaceutical ¹⁷⁷Lu-DOTA-HYNIC-iPSMA obtained in a healthy volunteer at different times.

FIG. 5. PET and SPECT image of the same patient of FIG. 4, with advanced metastatic prostate cancer who received both ⁶⁸Ga-PSMA-11 (PET, 1 h) and ¹⁷⁷Lu-DOTA-HYNIC-iPSMA (SPECT, 24 h).

FIG. 6. SPECT images for a patient with metastatic prostate cancer after the third cycle of treatment with ¹⁷⁷Lu-DOTA-HYNIC-iPSMA.

FIG. 7. Radiolabeling stability of [161Tb]Tb-DOTA-Hynic-iPSMA targeting agent using 100, 200, or 400 MBq of terbium-161 activity.

FIG. 8. Evolution of uncomplexed ¹⁶¹Tb when using different activities for radiolabeling.

FIG. 9. Evolution of radiolysis products over time according to the activity used for radiolabeling.

FIG. 10. Evolution of uncomplexed ¹⁶¹Tb with and without addition of EDTA.

FIG. 11. Radiolysis product with and without ascorbic acid.

FIG. 12. Saturation binding experiment of [161Tb]Tb-DOTA-Hynic-iPSMA.

FIG. 13. Membrane-bound fraction and internalized fraction of [161Tb]Tb-DOTA-Hynic-i PSMA.

FIG. 14. Efflux of [161Tb]Tb-DOTA-Hynic-iPSMA.

FIG. 15. Dose response curve of [161Tb]Tb-DOTA-Hynic-iPSMA on PC3-pip cells.

FIG. 16. Radio-TLC chromatograms of Ac-225 labeled DOTA-Hynic-iPSMA. Left: in the crude reaction mixture. Right: at EOS.

FIG. 17. Plot of fractions collected from the HPLC analysis at EOS applied with a gaussian fit to estimate the retention time and RCP of Ac-225-DOTA-Hynic-iPSMA.

FIG. 18. Radio-TLC chromatograms of Ac-225 labeled DOTA-Hynic-iPSMA. Left: in the crude reaction mixture. Right: at EOS.

FIG. 19. Plot of fractions collected from the HPLC analysis at EOS and 4 hr after EOS applied with a gaussian fit to estimate the retention time and RCP of Ac-225-DOTA-Hynic-iPSMA.

FIG. 20. Radio-TLC chromatograms of Ac-225 labeled DOTA-Hynic-iPSMA. Left: in the crude reaction mixture. Right: at EOS.

FIG. 21. Plot of fractions collected from the HPLC analysis at EOS applied with a gaussian fit to estimate the retention time and RCP of Ac-225-DOTA-Hynic-iPSMA.

FIG. 22A-22B. Plot of fractions collected from the HPLC analysis at EOS applied with a gaussian fit to estimate the retention time and RCP of Ga-68-DOTA-Hynic-iPSMA. Panel A) using a TELIX kit based approach and B) using a cassette based approach (Eckert & Ziegler).

### DETAILED DESCRIPTION OF THE INVENTION

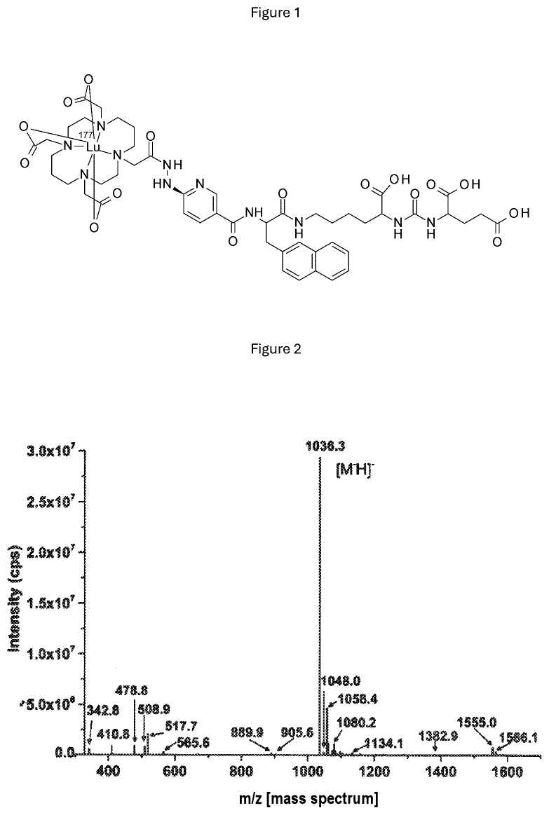

A novel lutetium-177 radiopharmaceutical that can inhibit prostate-specific membrane antigen (iPSMA) and which contains 1, 4, 7, 10-tetraazacyclododecano-N, N′, N″, N″″ -tetraacetic acid (DOTA) bound to the hydrazinonicotinamide (HYNIC) molecule, which is heterocyclic in nature, thus generating a rigid chemical structure that minimises the number of conformers and intramolecular hydrogen bonds, thereby resulting in a spatial orientation of the active site (Lys (Nal)—NH—CO—NH-Glu) in the molecule that favors biological recognition of the PSMA protein is presented for patent purposes. The novel radiopharmaceutical ¹⁷⁷Lu-DOTA-HYNIC-iPSMA accumulates in tumors overexpressing the protein PSMA with high affinity in vivo, thereby acting as a radiotherapeutic agent. The structure of the radiopharmaceutical to be patented (¹⁷⁷Lu-DOTA-HYNIC-iPSMA) is shown in FIG. 1.

Instead of being labelled with lutetium-177, the novel DOTA-HYNIC-iPSMA radiopharmaceutical molecule can also be radiolabelled with actinium-225, terbium-161 or gallium-67 or -68 as shown below and in the examples section.

Given the common knowledge that, given the heterocyclic nature thereof, pyridine has a dipolar moment and lower resonance energy than that of benzene (117 KJ·mol⁻¹ for pyridine versus 150 KJ·mol⁻¹ for benzene), as well as a shorter C—N bond (137 pm) compared with the value of 139 pm for the C—C bond in benzene and cyclohexane [Elschenbroich C. Organometallchemie, 6th ed., 2008, ISBN 3-8351-0167-6], the derivative ¹⁷⁷Lu-DOTA-HYNIC-iPSMA was designed and synthesized to obtain a poorly reactive and rigid chemical structure in the HYNIC region that minimises the number of conformers and intramolecular hydrogen bonds with respect to the derivatives ¹⁷⁷Lu-PSMA-617 and ¹⁷⁷Lu-PSMA-I&T. Table 1 below presents the comparative results for the molecular properties and optimal structural geometries of the different PSMA inhibitors. The ligands PSMA-617, DOTA-HYNIC-iPSMA, and PSMA-I&T were constructed taking into account the valence, bonding type, charge, and hybridisation. The minimum energies (obtained using augmented MM3) and lowest energy conformer (CONFLEX procedure) associated with the optimal geometry of the structures thereof were obtained using the CAChe Work System Pro software suite. The optimal geometrical structures were confirmed using quantum mechanical methods with the Schrodinger equation with MOPAC (molecular orbital), which calculates the heat of formation in water (COSMO). The lutetium complexes were constructed from said structures and the augmented MM3 (molecular mechanics) results, CONFLEX, and most stable and optimal geometric structure are presented for the radiopharmaceuticals Lu-PSMA-617 and Lu DOTA-HYNIC-iPSMA and Lu-PSMA-I&T. It can be seen from table 1 that the Lu DOTA-HYNIC-iPSMA molecule is more stable than Lu-PSMA-617 according to the energy of the most stable conformer (lower energy) and the lower total number of conformers, and Lu-DOTA-HYNIC-iPSMA forms fewer hydrogen bonds than Lu-PSMA-617 and Lu-PSMA-I&T. It should be noted that hydrogen bonds play a key role in spatial conformation as, although Lu-PSMA-I&T presents the lowest number of conformers with the lowest minimum energy, and the hydrophobic rings of the molecule are compromised in the conformation thereof when oriented, mainly due to said weak hydrogen interactions with the ¹⁷⁷Lu-DOTA ring (benzene-[m-iodo-phenol]-¹⁷⁷Lu-DOTA interactions). As such, the optimal structural geometries of the different radiopharmaceuticals presented in table 1 indicate a suitable spatial conformation for Lu-DOTA-HYNIC-iPSMA, which results in a spatial orientation (with no significant intramolecular interactions) for the active site (Lys (Nal)—NH—CO—NH-Glu) of the molecule that favors biological recognition by the protein PSMA. In other words, the three carboxyl groups of the Glu—NH—CO—NH-Lys fragment remain free to interact electrostatically with the peptide side chains at the active site of PSMA, the urea oxygen to coordinate to zinc, and the aromatic structure in Nal to couple to the active hydrophobic site in the enzyme. Moreover, in the structure of the radiopharmaceutical to be patented, HYNIC is not used as a molecule for chelation to the radiometal, whereas in other radiopharmaceuticals, HYNIC is used only as a binfunctional agent for labelling with ^{99m}Tc [Decristoforo C et al., ^{99m}Tc-EDDA/HYNIC-TOC: a new ^{99m}Tc-labelled radiopharmaceutical for imaging somatostatin receptor-positive tumours; first clinical results and intra-patient comparison with ¹¹¹In-labelled octreotide derivatives; 2000, J Nucl Med 27; 1318-25; Ferro-Flores G et al. Preparation and Evaluation of ^{99m}Tc-EDDA/HYNIC-[Lys3]-Bombesin for Imaging of GRP Receptor-Positive Tumours. Nucl Med Comm, 2006, 27:371-376; González-Vázquez A et al. Dosimetry and Biokinetics of ^{99m}Tc-EDDA/HYNIC-Tyr3-Octreotide Prepared from Lyophilized Kits. Appl Red Isot, 2006, 64:792-79; Ortiz-Arzate Z et al. Kit preparation and biokinetics in women of ^{99m}Tc-EDDA/HYNIC-E-[c(RGDfK)]₂for breast cancer imaging. Nucl Med Common, 2014, 35:423-32; Medina-Garcia V el al. A Freeze-Dried Kit Formulation for the Preparation of Lys²⁷ (^{99m}Tc-EDDA/HYNIC)-Exendin (9-39)/^{99m}Tc-EDDA/HYNIC-Tyr3-Octreotide to Detect and Benign Malignant Insulinomas. Nucl Med Biol, 2015, 42:911-916].

TABLE 1
MOLECULAR PROPERTIES OF THREE PSMA PROTEIN INHIBITING LIGANDS WITH TUMOR UPTAKE AND
RADIOTHERAPEUTIC EFFECT DEMONSTRATED IN CLINICAL STUDIES WHEN COORDINATED TO Lu-177

| CALCULATION | | | DOTA-HYNIC- | Lu-DOTA- | | |
|---|---|---|---|---|---|---|
| (kcal/mol) | PSMA-617 | Lu-PSMA-617 | IPSMA | HYNIC-IPSMA | PSMA-I&T | Lu-PSMA-I&T |
| Minimum energy (MM3) | 87.795 | −49.038 | 106.412 | −76.360 | 105.652 | −81.031 |
| Electrostatic charge | −20.447 | −197.887 | −10.061 | −206.795 | −22.131 | −210.822 |
| Hydrogen bond | 25.317 | 27.070 | 22.253 | 19.984 | 27.304 | 35.499 |
| van der Waals | 45.671 | 58.033 | 50.036 | 71.704 | 50.840 | 127.435 |
| Strength (stretch) | 6.994 | 10.184 | 7.398 | 26.970 | 9.471 | 50.100 |
| Angle | 16.360 | 36.203 | 20.704 | 84.579 | 20.504 | 143.558 |
| Bending strength | 0.937 | 1.196 | 1.117 | 4.282 | 1.180 | 8.849 |
| Dihedral | 14.232 | 32.247 | 15.863 | 42.282 | 20.508 | 83.716 |
| Improper torsion | 0.173 | 0.375 | 0.264 | 0.290 | 0.207 | 1.064 |
| Torsion strength | −1.288 | −1.925 | −1.159 | −3.269 | −1.803 | −6.234 |
| Bending bending | −0.154 | 0.841 | −0.003 | 3.044 | −0.426 | 8.513 |
| CONFLEX | 74.127 | −48.229 | 78.386 | −76.241 | 39.006 | −275.044 |
| Energy most stable | | | | | | |
| conformer | | | | | | |
| No. conformers stored | 1131 | 46 | 438 | 24 | 498 | 9 |
| MOPAC/PM5 | −715.945 | | −623.942 | | −1033.201 | |
| (heat of formation) | | | | | | |
| MOPAC/PM5/COSMO | −788.083 | | −698.534 | | −1136.015 | |
| (heat of formation in | | | | | | |
| water) | | | | | | |

| | |
|---|---|
| Optimal geometry for the radiopharmaceutical Lu-PSMA-617 | 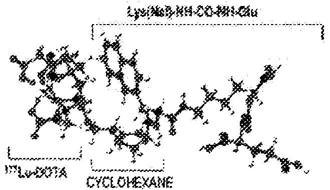 |
| Optimal geometry for the radiopharmaceutical Lu-DOTA-HYNIC-IPSMA | 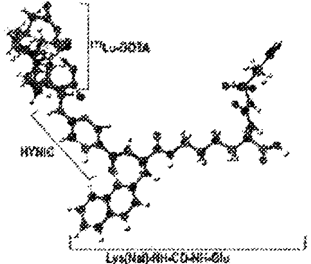 |
| Optimal geometry for the radiopharmaceutical Lu-PSMA-I&T | 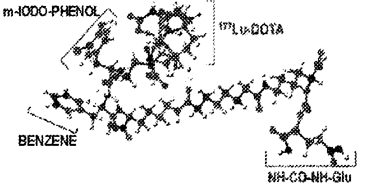 |

### Method for Preparing the Radiopharmaceutical of the Invention

The di-tert-butyl ester of glutamic acid was used initially to synthesise the molecule, said ester being reacted with carbonyldiimidazole (CDI) in the presence of triethylamine (TEA) to form the acylimizazole derivative, which was activated with methyl triflate (MeOTf) to react with(S)-tert-butyl-2-amino-6-(benzyloxycarbonylamino) hexanoate (Cbz-Lys-Ot-Bu), with subsequent deprotection of the Cbz by hydrogenolysis, thus giving the derivative Glu-Urea-Lys, which was reacted with the amino acid Fmoc-β-naphthyl alanine (HBTU/HOBt) in the solid phase (MBHA resin), followed by 6-Boc-hydrazinopyridin-3-carboxylic acid (Boc-HYNIC) in the presence of diisopropylethylenamine (DIPEA) and dimethylformamide (DMF) followed by addition of TFA. This latter addition step was repeated to introduce DOTA-tris (t-Bu ester) Finally, the compound was deprotected with TFA, purified by HPLC, and lyophilised. The final product was Glu—NH—CO—NH-Lys (β-naphthyl alanine)-HYNIC-DOTA (DOTA-HYNIC-iPSMA), which presented the expected mass spectrum shown in FIG. 2. Reverse-phase HPLC analysis of the lyophilised white solid showed a chemical purity of 98.8% for the compound.

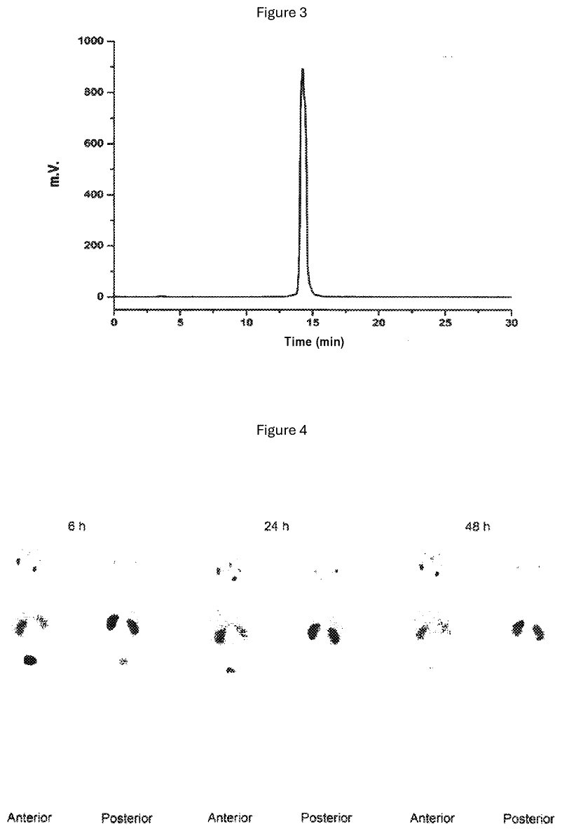

DOTA-HYNIC-iPSMA (0.6 mg) was formulated as a lyophilised pharmaceutical form containing 50 mg mannitol and 100 mg ascorbic acid. After reconstitution in 1.1 mL sodium acetate 1 M buffer solution pH 5.0 containing the sterile and pyrogen-free solution of lutetium-177 chloride (¹⁷⁷LuCl₃) and incubation in a dry bath at 95° C. for 30 minutes, said formulation gave a clear, aqueous solution of the compound to be patented ¹⁷⁷Lu-DOTA-HYNIC-iPSMA (FIG. 1) with a radiochemical purity of more than 98%, as determined by reverse-phase HPLC, which presents the radio-chromatogram shown in FIG. 3.

The radiopharmaceutical remains stable, with a radiochemical purity of more than 98% for more than 7 days post-labelling. In vitro stability tests in human serum show a serum protein binding of 6.5±1.8% and a high radiochemical stability (>98%). The affinity of ¹⁷⁷Lu-DOTA-HYNIC-iPSMA, as determined from saturation studies in cancer cells positive for the protein PSMA (LNCaP), showed a K_{d} of 6.33±2.69 nM and a maximum number of binding sites (Bₘₐₓ) Of 5.89±0.47 nM.

The compound did not present toxicity or adverse effects when administered at a dose of 40 mg/kg in balb-C laboratory mice. Biodistribution assays for ¹⁷⁷Lu-DOTA-HYNIC-iPSMA in nude mice with LNCaP-induced tumors showed an uptake in said tumors of 9.74±1.13% of the activity administered per gram of tissue (% ID/g) with a mainly renal elimination pathway.

To determine the biokinetics and dosimetry for the radiopharmaceutical, whole-body images were acquired for five healthy subjects at 20 mins and 6, 24, 48 and 120 h post-administration of ¹⁷⁷Lu-DOTA-HYNIC-iPSMA (185 MBq). The sequence of images was used to extrapolate the time-activity curves in each organ to adjust the biokinetic model and calculate the total number of disintegrations (N) that occurred in the source regions. The values of N were used in the OLINDA/EXM code to calculate the internal doses of radiation. The images in healthy volunteers showed fast clearance, with a half life of 1.1 h for the fast component (T_{1/2}α=ln2/0.614), 9.2 h for the first slow component (T_{1/2}β=ln2/0.075), and 79.6 h for the second slow component (T_{1/2} γ=ln2/0.008). Uptake and excretion is mainly renal, with lower hepatic uptake and high uptake in the parathyroid, salivary, and lachrymal glands. The average doses absorbed were 0.23, 0.28, 0.88, and 1.17 mGy/MBq for the spleen, liver, kidney, and salivary glands, respectively.

To evaluate the radiotherapeutic potential, between one and four cycles of ¹⁷⁷Lu-DOTA-HYNIC-iPSMA (3.7 or 7.4 GBq) were administered to 11 patients (mean age: 66 years; range: 45-86) every 8-10 weeks. The response was evaluated using images with ⁶⁸Ga-PSMA-11 PET/CT (radiopharmaceutical with a proven affinity in clinical practice and a high-resolution technique for the specific detection of metastatic prostate cancer lesions) and determining serum prostate-specific antigen (PSA) levels before and after treatment. Around 60% of patients exhibited a reduction in PSA and 70% a reduction in the number and size of metastatic lesions and/or in the uptake intensity of the radiopharmaceutical in the metastases and the primary tumor, as determined by imaging.

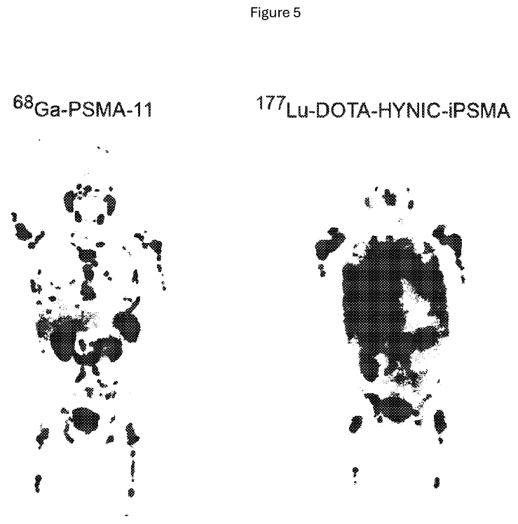

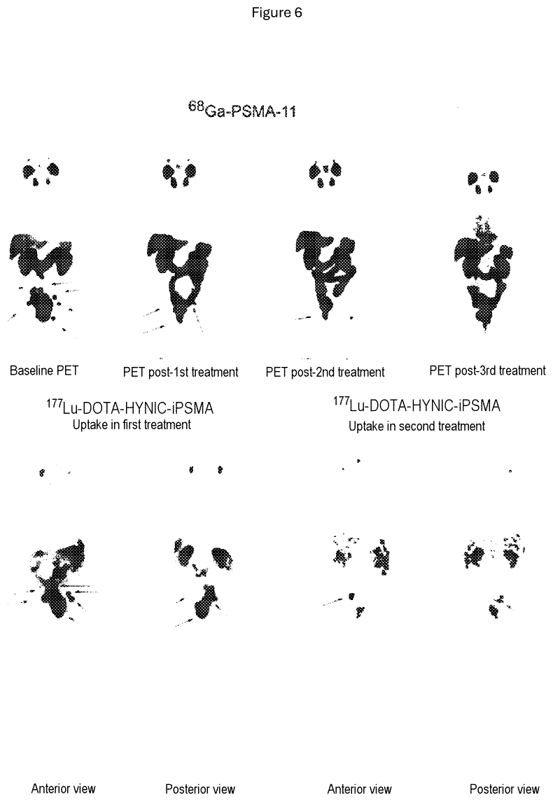

FIG. 4 shows a image for the SPECT image for the radiopharmaceutical ¹⁷⁷Lu-DOTA-HYNIC-iPSMA obtained in a healthy volunteer at different times. FIG. 5 shows a PET and SPECT image of the same patient with advanced metastatic prostate cancer who received both ⁶⁸Ga-PSMA-11 (PET, 1 h) and ¹⁷⁷Lu-DOTA-HYNIC-iPSMA (SPECT, 24 h), showing that both radiopharmaceuticals detect prostate cancer tumors and metastases, associated with overexpression of PSMA, thereby confirming the ability of the radiopharmaceutical ¹⁷⁷Lu-DOTA-HYNIC-iPSMA to detect the PSMA overexpressed in prostate cancer cells in vivo. Finally, FIG. 6 shows images for a patient with metastatic prostate cancer after the third cycle of treatment with ¹⁷⁷Lu-DOTA-HYNIC-iPSMA. Imaging-based follow-up with ⁶⁸Ga-PSMA-11 (PET) shows that the prostate cancer metastatic lesion sites (indicated with an arrow) decrease in size and number after each administration of ¹⁷⁷Lu-DOTA-HYNIC-iPSMA until complete elimination. Said image confirms and is the main evidence for the radiotherapeutic potential of ¹⁷⁷Lu-DOTA-HYNIC-iPSMA for the treatment of tumoral lesions overexpressing PSMA. In conclusion, ¹⁷⁷Lu-DOTA-HYNIC-iPSMA is obtained with the following features:
A radiochemical purity of greater than 98%.
The ability of the radiopharmaceutical to detect tumors overexpressing prostate-specific membrane antigen in vivo associated with a favorable spatial orientation of the active site (Lys (Nal)—NH—CO—NH-Glu) of the molecule induced by the presence of HYNIC bound to the DOTA molecule.
As a result of the molecular recognition associated with a favorable spatial orientation of the active site (Lys (Nal)—NH—CO—NH-Glu) of the molecule induced by the presence of HYNIC bound to the DOTA molecule and labeled with lutetium-177, the radiopharmaceutical ¹⁷⁷Lu-DOTA-HYNIC-iPSMA exhibits radiotherapeutic properties, as shown by the significant reduction in serum PSA levels and a decrease in the number and size of metastatic lesions observed in prostate cancer patients treated with ¹⁷⁷Lu-DOTA-HYNIC-iPSMA.

The following additional experimental examples were performed:

### Examples 1-3: ²²⁵Ac Labelling

600 μg DOTA-HYNIC-iPSMA was reconstituted in a 0.5M ascorbic acid buffer.

A ²²⁵Ac solution was eluted from a thorium generator by 0.04M HCL and with a specific activity (cf. Table 2), was added to the DOTA-HYNIC-iPSMA solution, resulting in a 1:1 labelling mixture of ascorbic acid buffer and HCL eluate resulting in a pH of the radiolabelling solution of between 4.5 and 5.0, incubated for 15 minutes at 95° C. by stirring. Incorporation was determined by radio-thin layer chromatography (TLC). The compound was purified on an Oasis HLB purification column or a C18 SepPak purification column, washed with 0.05 M sodium citrate, and eluted in absolute ethanol. The compound was then formulated to an activity concentration of 370 kBq/mL in formulation buffer (i.e. and sodium ascorbate diethylenetriamine pentaacetate DTPA) and the radiochemical purity (RCP) of the purified and formulated product was determined by radio-TLC and radio high performance liquid chromatography (HPLC). The compound was re-analysed by radio-HPLC 4 hours after the end of synthesis to assess stability.

TABLE 2
²²⁵Ac labelling conditions

| | Example 1 | Example 2 | Example 3 |
|---|---|---|---|
| Specific activity labeled on | 100.0 | 100.0 | 100.0 |
| (kBq/nmol) | | | |
| Activity at start of reaction | 0.65 | 1.5 | 5.0 |
| (MBq) | | | |
| Reaction time (min) | 15.0 | 15.0 | 15.0 |
| Reaction temperature (° C.) | 95.0 | 95.0 | 95.0 |
| pH in reaction | 5.0 | 5.0 | 5.0 |
| Incorporation, crude radio- | 94.5 | 89.5 | 94.0 |
| TLC (%) | | | |
| Purification column | Oasis HLB | C18 SepPak | C18 Seppak |
| Radiochemical purity (RCP), | 96.7 | 98.6 | 98.2 |
| EOS radio-TLC (%) | | | |
| Radiochemical purity (RCP), | 98.5 | 99.0 | 99.4 |
| EOS radio-HPLC (%) | | | |
| Radiochemical yield (%) | 27.8 | 77.6 | 51.0 |
| Specific activity in final | 94.5 | 89.5 | 94.0 |
| product (kBq/nmol) | | | |
| Activity concentration in | 370.0 | 370.0 | 740.0 |
| final product (kBq/mL) | | | |
| Compound concentration in | 3.9 | 4.1 | 7.9 |
| final product (nmol/mL) | | | |
| Radiochemical purity (RCP), 4 | — | 97.9 | — |
| H after EOS radio-HPLC (%) | | | |

Examples 1 to 3 show that DOTA-HYNIC-iPSMA was radiolabeled with ²²⁵Ac and showed incorporation of up to 89.5% in the crude reaction mixture by radio-TLC. For Example 2, the radiochemical purity (RCP) was 98.6% by radio-TLC and 99.0% in the final product determined by radio-HPLC. No major signs of instability were observed when the compound was re-analysed 4 hours after synthesis, resulting in an RCP of 97.9%. Based upon the radiolabeling data, the quality control release criteria of 295.0% purity and ≤5.0% of free ²²⁵Ac by radio-TLC are met.

### RESULTS

### Example 1

The compound was labeled with 650.0 kBq of Ac-225 and an incorporation of 94.5% was obtained determined by radio-TLC. The compound was purified on an HLB purification column, eluted in 250 μL of abs. EtOH and formulated to an activity concentration of 370 MBq/nmol and a compound concentration of 3.9 nmol/mL in formulation buffer. A radiochemical purity (RCP) in the final product of 96.7% was obtained determined by radio-TLC. A sample was withdrawn for radio-HPLC analysis and fractions of 30 seconds were collected and analysed on the well counter the day after when secular equilibrium was reached. An RCP of 98.5% was observed in the final formulation determined by radio-HPLC.

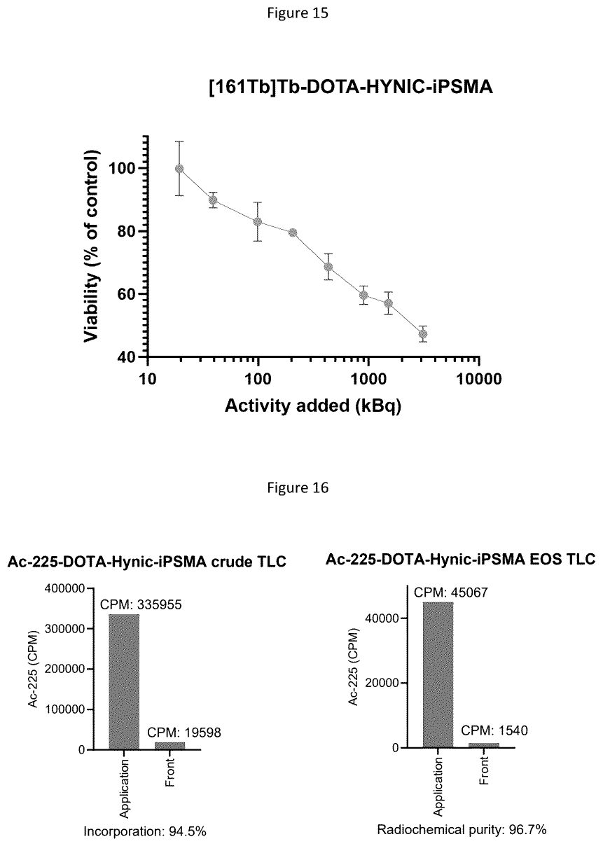

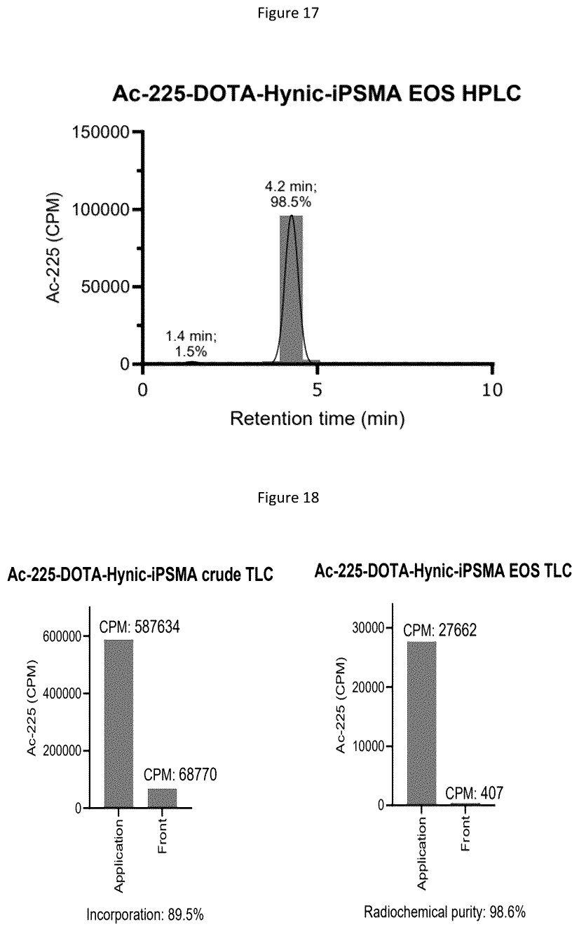

Due to some In-111 contamination of the fraction collected when the sample was re-run on HPLC 4 hr after EOS no data was obtained at this stability timepoint. The results are given in FIGS. 16 and 17.

### Example 2

The compound was labeled with 1.5 MBq Ac-225 and an incorporation of 89.5% was obtained determined by radio-TLC. The compound was purified on an C18 seppak purification column, eluted in 250 μL of abs. EtOH and formulated to an activity concentration of 370 MBq/nmol and a compound concentration of 4.1 nmol/mL in formulation buffer.

A radiochemical purity (RCP) in the final product of 98.6% was obtained determined by radio-TLC. A sample was withdrawn for radio-HPLC analysis and fractions of 30 seconds were collected and analysed on the well counter the day after when secular equilibrium was reached. An RCP of 99.0% was observed in the final formulation determined by radio-HPLC.

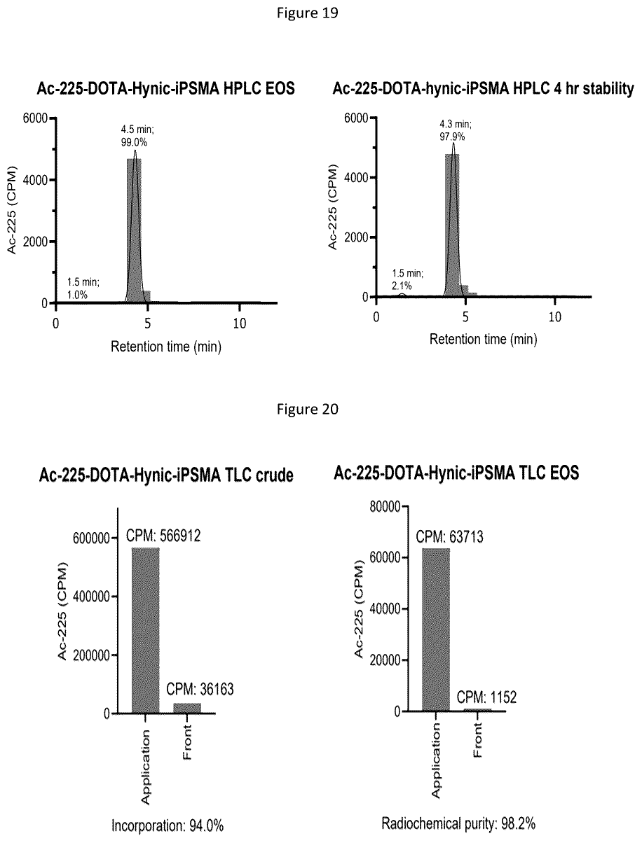

The compound was re-analysed by radio-HPLC 4 hr. after EOS and only minor degrees of instability were observed resulting in an RCP of 97.9%. The results are given in FIGS. 18 and 19.

### Example 3

The compound was labeled with 5.0 MBq Ac-225 and an incorporation of 94.0% was obtained determined by radio-TLC. The compound was purified on a C18 seppak purification column, eluted in 300 μL of abs. EtOH and formulated to an activity concentration of 740 MBq/nmol and a compound concentration of 7.9 nmol/mL in formulation buffer.

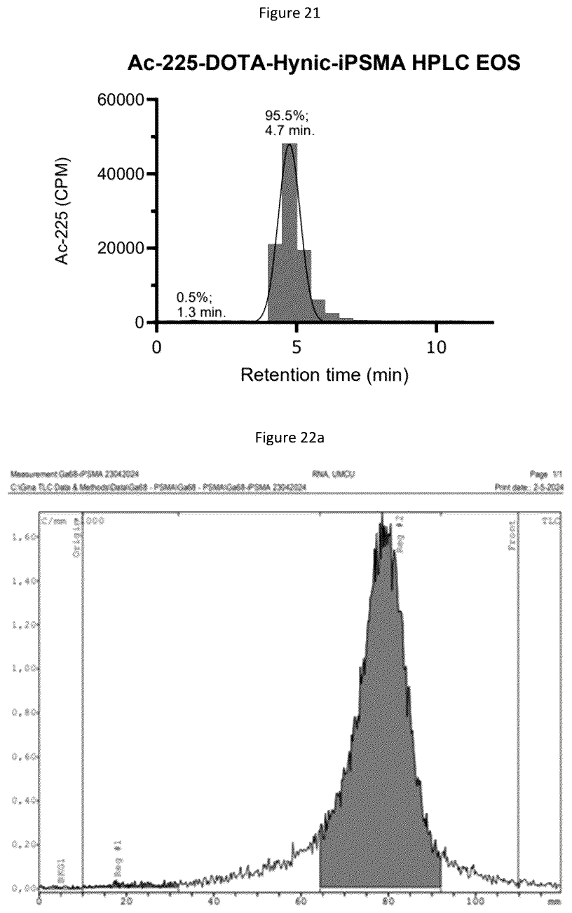

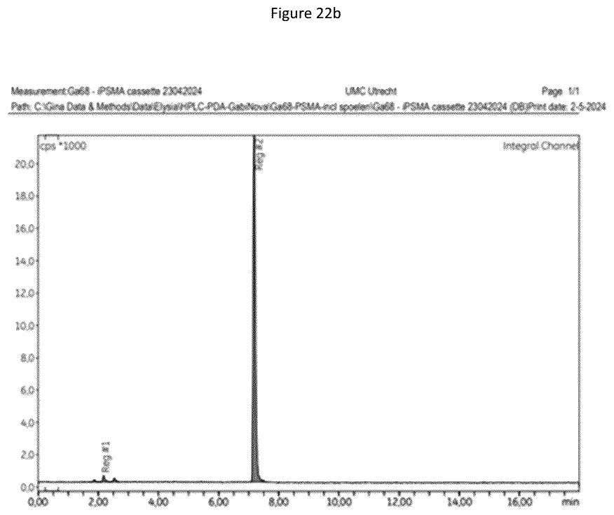

A radiochemical purity (RCP) in the final product of 98.2% was obtained determined by radio-TLC. A sample was withdrawn for radio-HPLC analysis and fractions of 30 seconds were collected and analysed on the well counter the day after when secular equilibrium was reached. An RCP of 99.5% was observed in the final formulation determined by radio-HPLC. The results are given in FIGS. 20 and 21.

Radiochemistry conditions and results from the 3 test labelings (Examples 1 to 3) are summarized in Table 1.

### Examples 4-7: ¹⁶¹Tb Labeling

For each example 600 μg DOTA-HYNIC-iPSMA was dissolved (reconstituted) in an ammonium acetate buffer as indicated in Table 3. To this solution, ¹⁶¹TbCl₃ solutions in an aqueous 0.05M HCl eluates as indicated in Table 2, were added from a Gadolinium-160 generator? (please give a ref.). The obtained reaction mixtures were heated at 90° C. for 30 minutes using a heating-block. The reaction mixtures were then let to cool down for 5 min. Examples 4 to 6 were immediately subjected to radio-UV-HPLC for quality control. To Example 7, 10 μL of ascorbic acid at 150 g/L was added along with 18 μL of EDTA 4 mM before being subjected to radio-UV-HPLC.

Radio-UV-HPLC analysis were performed using a Phenomenex Vydac C18 column (4 mL/min, λ=220 nm C18; 150 mm×4, 6 mm ×5 μm). HPLC conditions were: 0-2 min: 100% water in 0.1% TFA (A), 2-4 min: 100% ACN (B), 4-5 min: 100% B; 5-7 min: 100% A. The analytical HPLC system used was a JASCO system with ChromNAV software, a PU-2089 Plus quaternary gradient pump, a MD-2018 Plus photodiode array detector and Raytest Gabi Star detector.

For the evaluation of the radiolabeling stability, radio-UV-HPLC was repeated daily over a period of 4 days. Non-complexed ¹⁶¹Tb and radiolysis products, areas at the retention time of DOTA-HYNIC-iPSMA, solvent and all other peaks were integrated.

TABLE 3
¹⁶¹Tb labelling conditions

| | | Example | Example | Example | Example |
|---|---|---|---|---|---|
| | | 4 | 5 | 6 | 7 |
| Days since ¹⁶¹Tb production | | 12 | 12 | 12 | 15 |
| Activity at start of reaction | | 100 | 200 | 400 | 300 |
| (MBq) | | | | | |
| Volume ¹⁶¹TbC1₃ solution (16 μl) | | 16 | 32 | 64 | 150 |
| Molarity buffer (M) | | 0.4 | 0.4 | 0.4 | 2.0 |
| Volume buffer (μ) | | 200 | 200 | 200 | 300 |
| pH in reaction | | 5.1 | 5.1 | 5.1 | 5.5 |
| Reaction temperature (° C.) | | 90.0 | 90.0 | 90.0 | 90.0 |
| Reaction time (min) | | 30.0 | 30.0 | 30.0 | 30.0 |
| ascorbic | Volume (μl) | — | — | — | 10 |
| acid | Concentration (g/L) | | | | 15 |
| EDTA | Volume (μl) | — | — | — | 18 |
| | Concentration (mM) | | | | 4 |

| | | | | |
|---|---|---|---|---|
| Radiochemical purity (RCP), EOS | 97.36 | 96.14 | 95.74 | 95.00 |
| radio-HPLC (%) | | | | |

### Results for Examples 4 to 6

Radiochemical purities of [161Tb]Tb-DOTA-HYNIC-iPSMA were 97.36% (100 MBq—Example 4), 96.14% (200 MBq—Example 5) and 95.74% (400 MBq—Example 6). No purification was needed. RCP>98% might be achieved when using 161Tb closer to the production date.

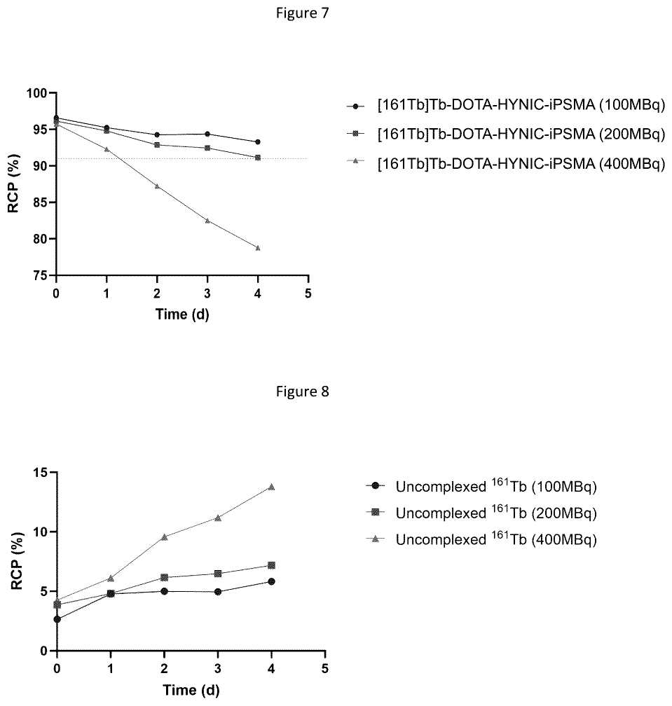

FIGS. 7, 8 and 9 showed the evolution of the radiolabeling stability, uncomplexed 161Tb and radiolysis products up to 4 days after radiolabeling.

As seen in FIG. 7, radiolabeling was stable up to 200 MBq. At higher activity (eg 400 MBq), radiolabeling was less stable and rapidly decrease.

Consequently, uncomplexed ¹⁶¹Tb increasing more rapidly at higher activity as displayed in FIG. 8. Addition of complexing agents significantly improves the final result.

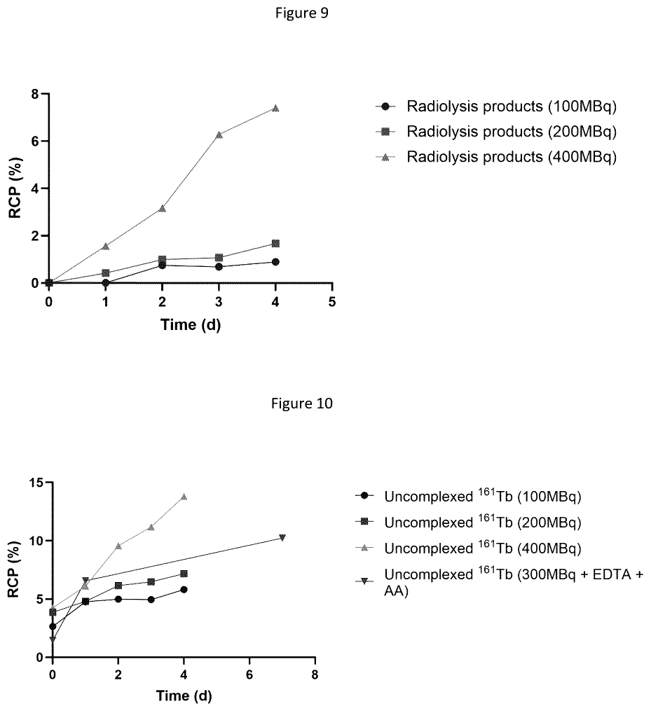

Finally, monitoring of radiolysis products showed that radiolysis occurs at activity as low as 400 MBq (FIG. 9). Therefore, anti-oxidants significantly improves the final result.

For Example 7, 300 MBq (150 μL) of ¹⁶¹Tb in HCl 0.05M were available. ¹⁶¹Tb was used 15 days after production. To deal with potential higher metallic contaminants, ammonium acetate buffer 2M, pH 5.5 was used (300 μL). Similar radiolabeling procedure than test 1 was followed. After cooling down, 10 μL of ascorbic acid at 150 g/L was added along with 18 μL of EDTA 4 mM.

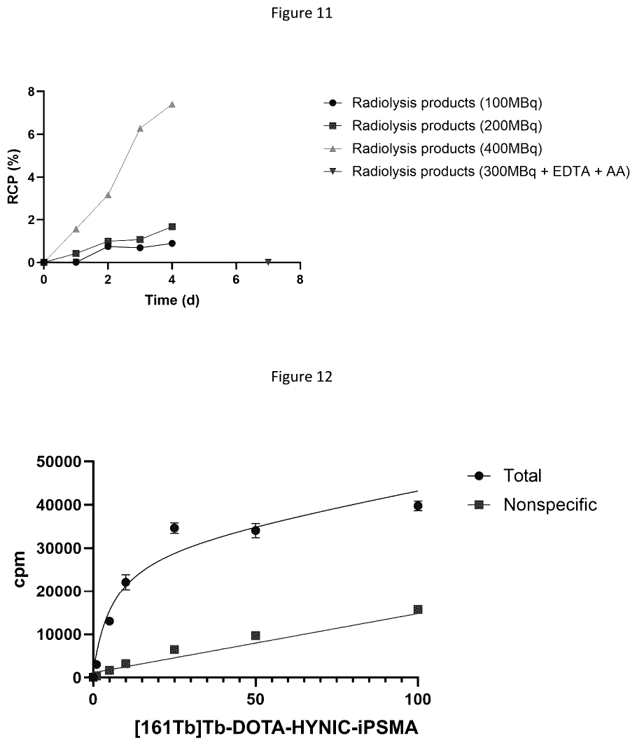

Radiochemical purity was 95.00% suggesting that using ¹⁶¹TbCl₃ 15 days after production is the latest limit. Interestingly, no radiolysis products were seen over a period of 7 days revealing that 10 μL ascorbic acid at 150 g/L is effective as quencher. However, uncomplexed ¹⁶¹Tb is still seen revealing that 18 μL EDTA 4 mM is not high enough (cf. FIGS. 10 and 11). This finding is confirmed by radio-HPLC showing uncomplexed ¹⁶¹Tb and 0.04 min later another peak attributed to [161Tb]Tb-EDTA (not shown). Therefore, higher concentration of EDTA may have to be added for effective complexation of unbound ¹⁶¹Tb and increase the shelf-life.

1 GBq of ¹⁶¹Tb produced 7 days before will be used. Radiolabeling will be performed in ammonium acetate buffer 0.4M, pH 5.1. The raw solution will be heated at 90° C. for 30 min and let cool down to room temperature for 5 min. 10 μL ascorbic acid at 150 g/L plus 10 μL EDTA 50 mM will be added next and the solution subjected to radio-HPLC.

### Example 8—In Vitro Assays

For all in vitro assays, PC3-pip cells were used. They were grown at 37° C., % CO2, in RPMI medium supplemented with 10% FBS, penicillin and streptomycin. Cells were seeded into 24-wells plates at the density of 2×105 cells per well, the day before the experiment.

### 1. AFFINITY

### Material and Methods

Plates were stored at 4° C. for 30 min in order to reduce cell processing and then incubated for 2 h with 250 μL of complete medium containing the radiolabeled peptide at increasing concentrations (1, 5, 10, 25, 50 and 100 nM) with and without the PSMA inhibitor PMPA used at the concentration of 10 μM). After incubation, cells were rinsed twice with ice cold DPBS (250 μL, 14190-144, Gibco). Then, cells were lysed with NaOH 1 M (750 μL) to collect the bound fraction. Radioactivity of each fraction was determined in a gamma-counter (Wizard2, PerkinElmer, USA). Affinity (Kd) was determined by nonlinear regression using Prism 10.2 software (GraphPad Software Inc., USA).

### Results

Non-linear regression provides a Kd value of 6.1 nM corresponding to 2.24 fmol/mg proteins (FIG. 12).

### 2. INTERNALIZATION ASSAY

### Material and Methods

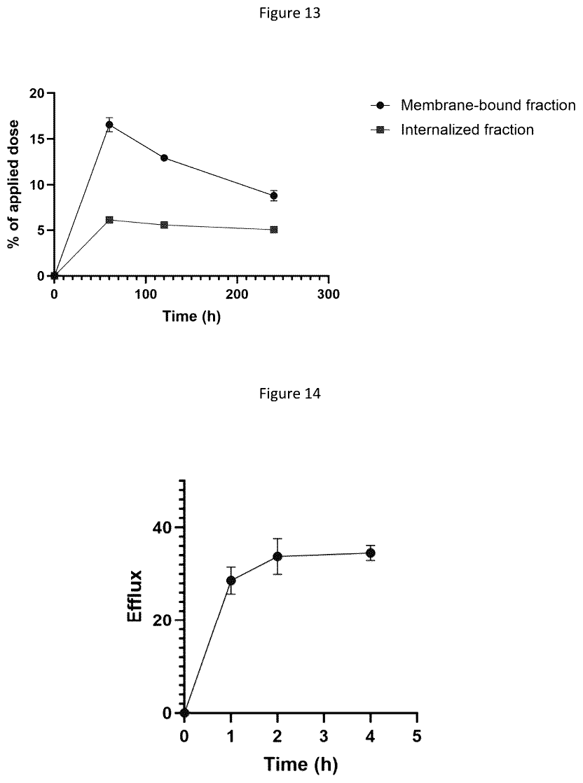

A total of 20 nM of [161Tb]Tb-DOTA-HYNIC-iPSMA in 250 μL of medium with and without 10 μM of the PSMA inhibitor PMPA was added in each well. Plates were incubated for 60, 120, or 240 min at 37° C., 5% CO2. Three minutes before the selected time point, plates were stored at 4° C. to stop the internalization process. Media were removed, and wells were rinsed three times with ice cold DPBS (250 μL). A total of 250 μL of sodium acetate (20 mM, pH 5) was added in each well twice and then collected in tubes after 5 min incubation. Then, cells were lysed with NaOH 1 M (750 μL) to collect the bound fraction. Radioactivity of each fraction was determined in a gamma-counter. Cell-associated radioactivity results are expressed as percentage of specific binding. The results are shown in FIG. 13.

### 3. EFFLUX STUDIES

### Material and Methods

A total of 20 nM of [161Tb]Tb-DOTA-HYNIC-iPSMA in 250 μL of medium was added in each well. Plates were incubated 30 min at 37° C., 5% CO2. Media were removed, and cells were rinsed with ice cold DPBS (250 μL). A total of 250 μL of sodium acetate (20 mM, pH 5) was added in each well and removed after 5 min of incubation. Cells were rinsed again with ice cold DPBS (250 μL). Then, 250 μL of medium were added in each well and plates were incubated for 60, 120 or 240 min at 37° C., 5% CO2. Media were collected in tubes, and each well was rinsed twice with ice cold DPBS (250 μL). Then, cells were lysed with NaOH 1 M (750 μL) to collect the bound fraction. Radioactivity of each fraction was determined in a gamma-counter. Results are expressed as percentage of total binding. The results are given in FIG. 14.

### 4. THERAPY STUDY

### Material and Methods

2500 cells per well were seeded the day before the experiment in 96 well plates in RPMI supplemented with 10% FBS and antibiotics. The medium was then removed and increasing activities of [161Tb]Tb-DOTA-HYNIC-iPSMA (0, 0.1, 0.2, 0.5, 1, 2, 5, 10 and 20 MBq/mL) was added to the wells in medium without FBS for 4 h at 37° C., 5% CO2.

At the end of incubation the radiopharmaceutical was removed and cells were recultured for three additional days in RPMI supplemented with 10% FBS and antibiotics. At the end of the three, cell viability was evaluated using Cell Titer glo according to manufacturer's instructions. Luminescence was recorded using a multiplate reader (BMG Labtech). Results were express in percentage of untreated cells. To correct the results to the exact activity added in each well, standards containing the same activity than the wells were gamma counted owing the normalization factor of 23.97 cpm/Bq of our gamma counter (Perkin Elmer, Wizard2). Standards were counted until they fall into the acceptance range of ¹⁶¹Tb of our device (1000-1.7.106 cpm). IC50 was derived by fitting the curve using the «[inhibitor] vs normalized response »equation using GraphPad software v10.2. The results are given in FIG. 15. [161Tb]Tb-DOTA-HYNIC-iPSMA is able to inhibit cell viability in a (log) dose-dependent manner. Curve fitting showed that the IC50 is 1595 kBq, 95% CI [1319-1937 kBq].

### Example 9—Optimisation of Lutetium-177 Radiolabeling of DOTA-HYNIC-iPSMA.

600 μg of precursor was mixed with 50 mg/mL mannitol, and 50 mg/mL ascorbic acid dissolved in 1.1 mL acetate buffer (pH=5.0-5.5). The precursor solution was labelled with up to 10 GBq of ¹⁷⁷Lu-LuCl₃ solution, applying 85° C. for 10 minutes. The lutetium-177 radiolabelled DOTA-HYNIC-iPSMA obtained had a radiochemical purity of above 95% measured by HPLC.

The conditions and results are depicted in the Table below:

| | |
|---|---|
| Precursor [mg] | 0.6 |
| mannitol [mg] | 50 |
| ascorbic acid [mg] | 50 |
| acetate buffer [mL] | 1.1 |
| Lu-177 [mL] | 0.33 |
| Total volume | 1.4 |
| Synthesis method | 85° C., 10 minutes |
| Starting activity [GBq] | 9.5 |
| Appearance | Pass |
| pH | 4.9 |
| RCP (M2) | 98 |
| RCI (M2) | 105 |

### Example 10—Optimisation of Gallium-68 (or Gallium-67) Radiolabeling of DOTA-HYNIC-iPSMA

In this experiment the kit-based approach and cassette based approach were compared using the set-up indicated below for each of them. From the results (cf. FIGS. 16 a and b) we can derive that both approaches work well but the cassette based approach is more effective.

Kit Protocol:

Dissolve 50 microgram of DOTA-HYNIC-iPSMA (Telix) in 1 ml acetate buffer (Telix) and elute ⁶⁸GaCl₃ generator (Eckert & Ziegler) into said mixture with HCl, swivel and incubate for 10 minutes at 95° C. in a heating block, swivel and let cool (at room temperature) for 10 minutes.

Cassette Protocol (Modular Lab Easy Radiosyntheser-Eckert & Ziegler):

Dissolve 50 microgram of DOTA-HYNIC-iPSMA (Telix) in 1 ml acetate buffer (Telix) 2.3 ml ammonium acetate (Eckert & Ziegler) and 0.6 ml ethanol/water solution (Eckert & Ziegler) in a reaction vial. Eluting the ⁶⁸GaCl₃ generator (Eckert & Ziegler) and trapping the Gallium on an SCX column. Eluting said gallium from the column into said reaction vial with NaCl/HCl solution (Eckert & Ziegler. Heat the mixture for 10 minutes at 90° C. and purify over a CM ceramic column. Optionally it can be sterilized using known means.

1. A radiopharmaceutical with the chemical formula DOTA-HYNIC-iPSMA defined by structure (I):

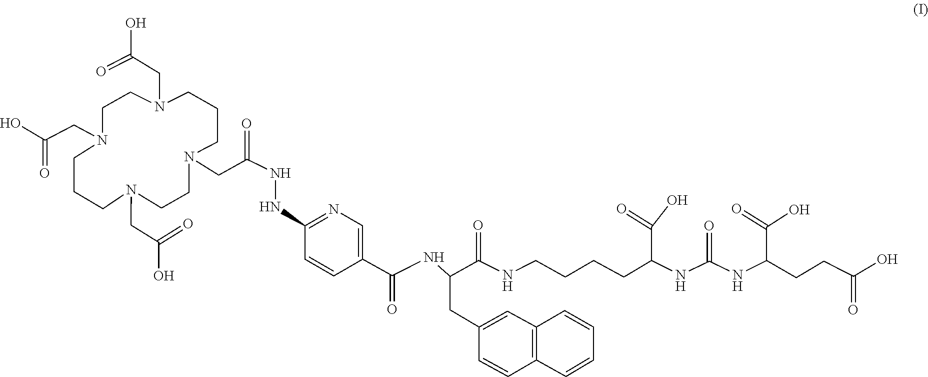

2. The radiopharmaceutical according to claim 1, radiolabelled with Lutetium-177 defined by structure (II):

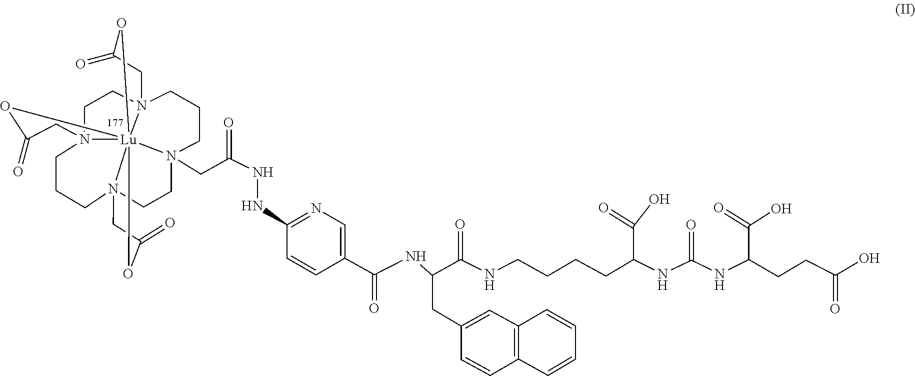

3. The radiopharmaceutical according to claim 1, radiolabelled with actinium-225 defined by structure (III):

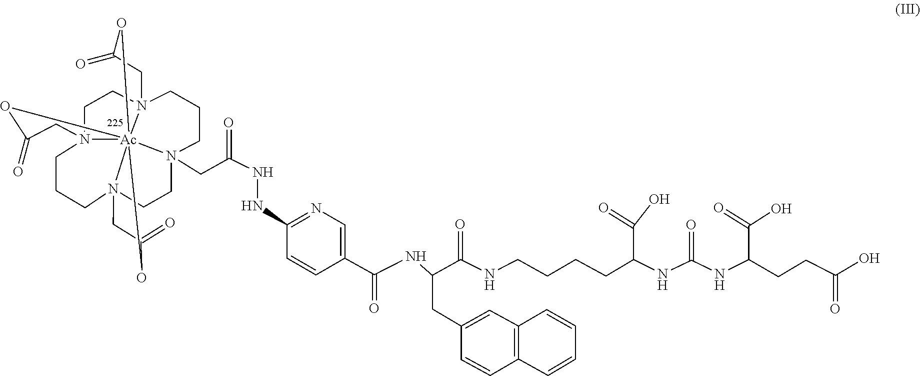

4. The radiopharmaceutical according to claim 1, radiolabelled with terbium-161 defined by structure (IV):

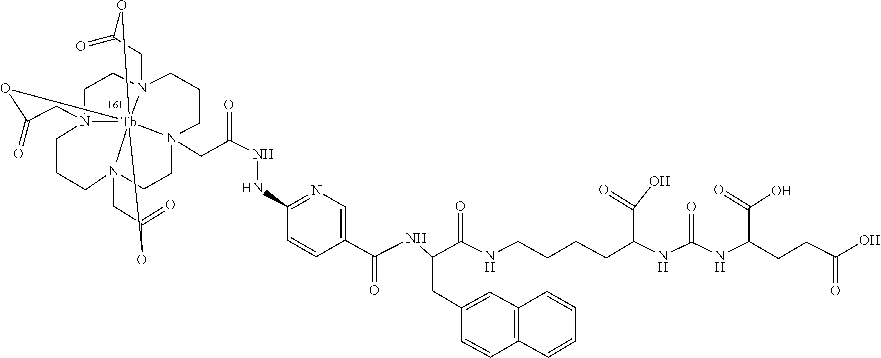

5. The radiopharmaceutical according to claim 1, radiolabelled with gallium-68 defined by structure (V):

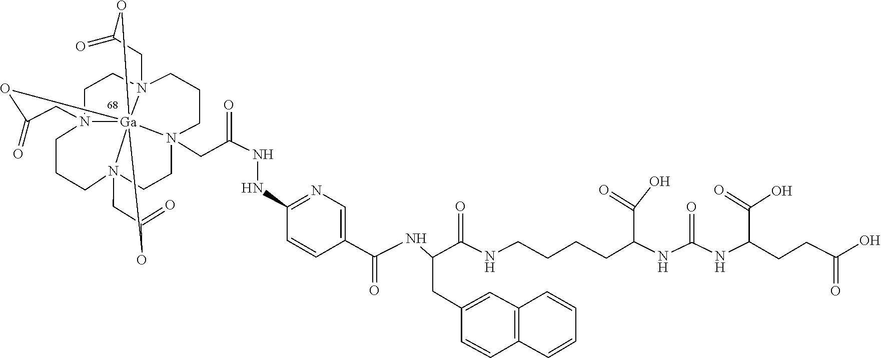

6. A radiopharmaceutical composition characterised by comprising a radiopharmaceutical such as that claimed in claim 1.

7. A radiopharmaceutical such as that claimed in claim 1 for use as a radiotherapeutic agent in tumors that overexpress the protein PSMA (prostate-specific membrane antigen).

8. The radiopharmaceutical according to claim 7, wherein said cancer is selected from the group consisting of: prostate cancer, salivary gland cancer, glioblastoma, thyroid cancer, clear cell renal carcinoma, renal cell carcinoma, hepatocellular carcinoma, lung cancer, adenoid cystic carcinoma of the parotid, and breast cancer.

9. An imaging method, comprising the steps of:
i) providing the DOTA-HYNIC-iPSMA radiopharmaceutical according to claim 1 radiolabelled with lutetium-177, actinium-225, terbium-161, or gallium-68,
ii) administering to a subject a diagnostic amount of said radiolabeled targeting agent; and,
iii) detecting said radiolabeled radiopharmaceutical when bound to its target PSMA using PET, SPECT, PET/CT or SPECT/CT imaging methods.

10. The imaging method according to claim 9, wherein said radiolabel is lutetium-177 and wherein the imaging method used is SPECT or SPECT/CT imaging.

11. The imaging method according to claim 9, wherein said radiolabel is actinium-225 and wherein the imaging method used is SPECT or SPECT/CT imaging.

12. The imaging method according to claim 9, wherein said radiolabel is terbium-161 and wherein the imaging method used is PET, PET/CT, SPECT or SPECT/CT imaging, preferably SPECT or SPECT/CT imaging.

13. The imaging method according to claim 9, wherein said radiolabel is gallium-68 and wherein the imaging method used is PET or PET/CT imaging.

14. A method of detecting a tumour or cancer, comprising the steps of:
i) providing a DOTA-HYNIC-iPSMA radiopharmaceutical according to claim 1 radiolabeled with lutetium-177, actinium-225, terbium-161, or gallium-68,
ii) administering to a subject a diagnostic amount of said radiolabeled DOTA-HYNIC-iPSMA; and,
iii) detecting binding of said radiolabeled DOTA-HYNIC-iPSMA to its PSMA target using PET, SPECT, PET/CT, or SPECT/CT imaging methods.

15. The method according to claim 14, wherein said detection is used for:
(i) initial staging of prostate cancer into intermediate, unfavourable, high, or very high-risk prostate cancer;
(ii) detecting suspected recurrence of prostate cancer and/or detection of metastasis;
(iii) selection for radiotherapeutic treatment such as with Lutetium (177Lu) vipivotide tetraxetan (Pluvicto);
(iv) monitoring prostate cancer for progression into Non-Metastatic or Metastatic Castration-Resistant Prostate Cancer (nmCRPC or mCRPC);
(v) determining response to (radio) therapy;
(vi) real-time imaging or diagnosis in a subject, such as for use in PET scans for tracking slow biological processes or progression of those over a period of time, optionally wherein the real-time imaging or diagnosis is carried out by a computer or processor and hence is directed by a system for in vivo diagnosis or for in vivo imaging in a subject, said system comprising a processing means (processor) configured to carry out the steps of receiving real-time imaging data and presenting said imaging data in real-time to a user, thereby enabling in vivo imaging or in vivo diagnosis of said subject; or (vii) personalised medicine.

16. The method according to claim 14, wherein said detection method is used to replace the need for taking a prostate biopsy or is used in SPECT, SPECT/CT, PET or PET/CT scan with MRI in clinically significant or intermediate favourable prostate cancers, or in MRI for active surveillance of prostate cancer.

17. A method for treatment of cancer, comprising the step of administering an effective amount of DOTA-HYNIC-iPSMA according to claim 1 radiolabelled with lutetium-177, actinium-225 or terbium-161 to a patient in need thereof.

18. The method according to claim 17, wherein said cancer is over-expressing PSMA.

19. The method according to claim 17, wherein said cancer is selected from the group consisting of prostate cancer, salivary gland cancer, glioblastoma, thyroid cancer, clear cell renal carcinoma, renal cell carcinoma, hepatocellular carcinoma, lung cancer, adenoid cystic carcinoma of the parotid, and breast cancer.

20. The method according to claim 17, wherein said cancer has been detected using a method comprising the steps of:
i) providing a DOTA-HYNIC-iPSMA radiopharmaceutical according to claim 1 radiolabeled with lutetium-177, actinium-225, terbium-161, or gallium-68,
ii) administering to a subject a diagnostic amount of said radiolabeled DOTA-HYNIC-iPSMA; and,
iii) detecting binding of said radiolabeled DOTA-HYNIC-iPSMA to its PSMA target using PET, SPECT, PET/CT, or SPECT/CT imaging methods.
